(19) United States
(12) Patent Application Publication
(10) Pub. No.: US 2025/0059126 A1
(43) Pub. Date: Feb. 20, 2025

(54) UTILIZATION OF POLYETHYLENE-CONTAINING MIXTURES TO FORM LONG-CHAIN ALKYL DICARBOXYLIC ACIDS BY WAY OF OXIDATIVE DEGRADATION

(71) Applicant: UNIVERSITÄT KONSTANZ, Konstanz (DE)

(72) Inventors: Manuel Haeussler, Konstanz (DE); Lukas Odenwald, Konstanz (DE)

(21) Appl. No.: 18/722,127

(22) PCT Filed: Dec. 20, 2022
(86) PCT No.: PCT/EP2022/086978

§ 371 Date: Jun. 20, 2024

(30) Foreign Application Priority Data
Dec. 20, 2021 (DE) 10 2021 133 861.1

(51) Int. Cl. C07C 51/215; C07C 51/44; C07C 51/47; C07C 51/48; C08J 11/16

(52) U.S. Cl. CPC C07C 51/215; C07C 51/44; C07C 51/445; C07C 51/47; C07C 51/48; C08J 11/16; C08J 2400/30

(57) ABSTRACT

The present invention relates to a process for producing a mixture that is insoluble in water of a homologous series of a number of various long-chain (≥C₈) alkyl dicarboxylic acids, more particularly α,ω-n-alkyl dicarboxylic acids, by oxidative degradation of polyethylene (PE)-containing mixtures with oxygen, and to the mixtures or compositions obtainable therefrom and to their uses.

The present invention relates to a process for producing a mixture that is insoluble in water of a homologous series of a number of various long-chain (≥C₈) alkyl dicarboxylic acids, more particularly α,ω-n-alkyl dicarboxylic acids, by oxidative degradation of polyethylene (PE)-containing mixtures with oxygen, and to the mixtures or compositions obtainable therefrom and to their uses.

In 2020, plastics production was estimated at 400 million tons worldwide, of which 15 million tons were produced in Germany alone. The most important class of plastic here is polyethylene, which with around 30% accounts for the largest share. This class is divided roughly equally into high-density polyethylene and low-density polyethylene (LDPE). The products made from it are very versatile and ubiquitous, but the most significant in terms of numbers are (disposable) packaging products such as various films or hollow bodies. This results in enormous quantities of plastic waste every year for the recycling of which there are currently only inadequate technical and economic solutions. As a result of this recycling problem such waste is increasingly being released into the environment. Due to the high mechanical and chemical stability of plastics this leads to the persistence of the materials in nature with the well-known devastating consequences for the environment. In addition, this also represents an enormous waste of finite resources and raw materials in purely economic terms.

In principle, plastic waste can be utilized using various approaches such as mechanical, raw material or energy recovery. In Germany, material recycling is currently the main approach. Here, the waste products are sorted, cleaned, shredded and melted down again. This type of recycling processes used plastics in order to provide a secondary raw material and regranulates for new plastic products. It is important that the polymers are as pure as possible and that their chemical structure is not changed during this process, as both factors are decisive for the quality of the material. However, due to impurities, the presence of other substances (such as dyes) the lack of options for separating the different types of plastic by type and ultimately the unavoidable progressive material damage caused by mechanical and thermal stress this recycling process is only feasible for around 40% of all plastic waste produced. In addition, the secondary products produced in this way are of a lower quality compared to the respective virgin material and are therefore often not suitable for the original applications. Due to a lack of better alternatives, this means that the remaining around 50% of all plastic waste in Germany has to be utilized energetically, i.e. incinerated, which currently does not result in a closed material cycle for plastics.

For these reasons, in recent years feedstock recycling has been increasingly discussed as another promising recycling method for plastic waste. In this process the plastics are chemically broken down into smaller components that can be much better separated and purified thereby ensuring access to materials of virgin quality. The prerequisite for raw material recycling is a given reactivity of the plastic for chain scission in order to enable efficient and selective extraction of the building blocks.

While this is primarily the case for polyesters such as PET for example and economic chemical recycling already exists the high chemical stability of HDPE in particular is extremely problematic in terms of feedstock recycling. So far thermal pyrolysis has established as the only notable process with sufficient activity for the degradation of HDPE. To achieve sufficient activity this reaction is carried out under harsh conditions of typically around 600° C. and as a result low selectivity. Thereby a petroleum-like mixture of hydrocarbons with a correspondingly low value is produced, making the economic viability of the process dependent on other factors. The actual value creation takes place in subsequent steps in which the pyrolysis oil is used as a substitute for crude oil in petrochemical processes and therefore depends directly on the continued existence of this industry in its current form.

In summary the above statements show that the current state of the art lacks long-term economic and at the same time environmentally compatible options for the utilization of HDPE waste in addition to infrastructurally demanding mechanical recycling. Due to the rapidly increasing quantities of this waste that are to be expected worldwide in the short and medium term over the next few decades a solution to this recycling problem is urgently required.

Processes for the oxidative degradation of polyolefins to produce α,ω-n-alkyl dicarboxylic acids have already been described in the state of the art. However, these have the disadvantage that very strong oxidizing agents, such as concentrated nitric acid, must be employed, which entail both ecological problems (formation of environmentally harmful nitrogen oxides) and enormous requirements with regard to the reactors used (corrosion). In the chemical recycling of polyolefins, the relative stability of the substrates follows the following trend: HDPE >>LDPE >>PP. Accordingly, the degradation of HDPE into small molecules represents the greatest challenge in the group of polyolefins. Despite the use of highly reactive nitric acid the reactivity of the processes described in the state of the art is sometimes not sufficient to efficiently degrade the inert HDPE, which is why only the significantly more labile substrates LDPE and PP are preferably employed. For example US 2020/102440 A1 and WO 2021/119389 A1 disclose the oxidative degradation of plastic waste in particular LDPE and PP waste in concentrated nitric acid to form mixtures of α,ω-n-alkyl dicarboxylic acids with typical average chain lengths in the range from C₅ to C₆. Furthermore, from E. Backström et al, Ind. Eng. Chem. Res., 2017, 56, 14814-14821 a microwave-assisted process for the oxidative degradation of only LDPE to short-chain dicarboxylic acid mixtures (mainly C₄-C₅) in nitric acid is already known. These short-chain dicarboxylic acid mixtures have the disadvantage that many highly efficient competing processes exist for the production of these compounds and the products from the processes therefore have a low economic value.

Furthermore, processes for the oxidative functionalization of polyethylenes, in particular polyethylene waxes, with oxygen (EP 0 890 583 A1, EP 3 470 440 A1) and/or ozone (EP 0 296 490 A2, GB 951 308 A) are known. The functionalization of polymers with oxygen-containing groups is primarily intended to improve the polarity and thus the surface properties of the polymers employed in various applications. The products obtained in this way are designated as oxidized PE waxes. These are used for example in dispersions of printing inks and paints, the coating of paper, as lubricants in plastics processing, in metal processing or as adhesives and sealants. Low-molecular polyethylenes (Mw<20 kg/mol) of high density (up to 0.97 g/cm3) are preferably used as the starting material in form of virgin material (e.g. EP 3 470 440 A1). In addition, the typically obtained oxidized polyethylene-like products only exhibit low acid numbers (<40 mg KOH/g) compared to the present invention since the focus here is on the functionalization of the existing polyethylene chain thus a completely different objective is pursued and there is also no recycling claim. In addition, U.S. Pat. No. 6,852,893 B2 and U.S. Pat. No. 8,487,138 B2 disclose for example processes for the oxidation of low molecular weight PE waxes, cycloalkanes and alkyl-substituted aromatics using N-hydroxyphthalimide (NHPI). The focus here is on the use of this organocatalyst to accelerate various known oxidation reactions using different hydrocarbons as starting materials. For example, the production of various oxidation products containing short-chain dicarboxylic acids from different hydrocarbons containing alkanes or PE waxes is described here. Neither polymers are described as starting materials nor the use of secondary raw materials and thus any recycling intention.

In a specific embodiment, GB 951 308 A describes the oxidation of very fine HDPE powder in a fluidized bed reactor using ozonized oxygen to produce acidic oxidized PE waxes for use as polishing wax for example. The decisive disadvantage and limitation of the process are the high requirements of the HDPE employed, which must be provided free of foreign matter as a fine powder and oxidized below the softening temperature in order to enable the reaction to be carried out in the required highly mixed fluidized bed reactor with ozonized oxygen. This reaction process is only economically viable for freshly produced HDPE which is obtained directly as a powder from synthesis and is therefore inherently not suitable for recycling plastic waste in the form of melt-processed bodies. These would first have to be cooled below their glass temperature (approx. −70 to −100° C.) with high energy input in order to be able to provide the powder for example via cryogenic grinding.

Accordingly, the object of the present invention is to provide a process that recycles HDPE-containing waste in an environmentally friendly, cost-effective, efficient and sustainable manner into sought-after alkyl dicarboxylic acids, in particular α,ω-n-alkyl dicarboxylic acids.

The task described above is solved by the embodiments of the present invention as set forth in the claims.

In particular, the invention provides a process for preparing a water-insoluble mixture of a homologous series of a plurality of different α,ω-n-alkyl dicarboxylic acids (linear α,ω-alkyl di-carboxylic acids) having a carbon chain length of at least C₈ starting from polyethylene-containing mixtures by oxidative degradation with an oxygen-containing reaction gas, comprising:
providing a polyethylene-containing mixture which has a total polyethylene content of at least 50% by mass with an HDPE content of at least 5.0% by mass, the polyethylenes contained each having a weight-average molecular weight (Mw) of at least 20,000 g/mol, and
heating the mixture provided to a temperature in a range from above the melting point of the polyethylene-containing mixture to 300° C. in an oxygen-containing reaction gas consisting of at least 5% by volume of oxygen, at a process pressure of from 1 to 100 bar and a reaction time of from 0.1 to 16 hours in order to oxidatively decompose the polyethylene-containing mixture,
   wherein the resulting product mixture of a plurality of different linear α,ω-alkyl di-carboxylic acids with a carbon chain length of at least C₈ has an acid number of at least 100 mg KOH/g. In the present invention oxidative degradation of the polyethylene or HDPE occurs above the melting point which significantly simplifies the requirements for the mixture employed and thus also allows the use of secondary raw materials for the first time. Until now, it has not been possible to control the significantly more active reaction above the melting point in such a way that long-chain α,ω-n-alkyl dicarboxylic acids can be obtained with high selectivity. In the present invention this could now be achieved for the first time without high requirements to the substrate (starting material) or mixing in the reactor which thus allows the feedstock recycling of HDPE-containing plastic waste by means of a robust and economic oxidative degradation reaction.

As an alternative to the previously established feedstock recycling methods the present invention describes a process for the oxidative degradation of polyethylene chains to produce a water-insoluble mixture of a homologous series of a plurality of different long-chain α,ω-n-alkyl di-carboxylic acids. By using reactive oxygen for example provided by the use of compressed ambient air the otherwise chemically inert PE waste can be degraded under particularly mild conditions and therefore oxidatively recycled into chemicals with a significantly higher value (chemical “upcycling”).

Surprisingly, with the process of the present invention, HDPE as the most demanding substrate of all polyolefins for oxidative recycling can also be recycled to long-chain dicarboxylic acids with a carbon chain length of at least C₈ and preferably an average carbon chain length ≥C₈ using atmospheric oxygen as an oxidizing agent. The decisive factor here is to control the highly active oxidation above the melting point by understanding the process parameters in such a way that no complete oxidation and thus combustion to CO₂ takes place but the reaction stops at the level of long-chain α,ω-n-alkyl dicarboxylic acids as degradation products.

Despite the wide range of existing applications these products can still only be produced at comparatively high cost from plant-based raw materials or using multi-stage petrochemical processes. One example of an α,ω-n-alkyl dicarboxylic acid with particular economic relevance is 1,12-dodecanedioic acid (DDDA) which serves as a monomer building block for the synthesis of polyamides and polyesters. For example, polycondensation of DDDA with 1,6-hexamethylenediamine produces the plastic polyamide 612 (PA 612) which is used in various applications, e.g. bristles for toothbrushes, pipes, conduits, etc. Accordingly, the market volume for this special DDDA is high and already amounted to around USD four billion in 2020. The synthesis of virgin DDDA is currently carried out via a cost-intensive, environmentally harmful and non-sustainable multi-stage synthesis route starting from 1,3-butadiene (again obtained from natural gas or coal) using various acids and reactants. Other economically important α,ω-n-alkyl dicarboxylic acids include 1,10-decanedioic acid (sebacic acid) which is also used intensively for the synthesis of polyamides and polyesters and 1,9-nonanedioic acid (azelaic acid), which is used in pharmaceuticals and creams both of which are produced from unsaturated fatty acids and therefore vegetable oils.

According to the present invention, however, it is possible starting from chemically inert HDPE-containing plastic waste to provide in an environmentally friendly, cost-effective, efficient and sustainable manner a composition consisting essentially of a water-insoluble mixture of a homologous series of at least 10 different linear α,ω-alkyl dicarboxylic acids having a carbon chain length in the range from C₈ to C₃₄ whereby the proportion of water-soluble compounds and compounds having a carbon chain length of C₃₅ or more is less than 10% by mass.

The mixtures or compositions obtained according to the invention can advantageously be used either directly for the production of polymers such as in the synthesis of polyamides and polyesters or for the production or isolation of the respective pure linear α,ω-alkyl dicarboxylic acids with a carbon chain length in the range from C₈ to C₃₄ contained in the mixture.

The process according to the invention and the mixtures or compositions obtainable therefrom are described in more detail below.

The process according to the invention relates to the production of water-insoluble α,ω-n-alkyl dicarboxylic acids with a carbon chain length of at least C₈, starting from PE-containing mixtures characterized by a total polyethylene content of at least 50% by mass comprising an HDPE content of at least 5.0% by mass, the polyethylenes contained each having a weight-average molecular weight (Mw) of at least 20,000 g/mol by oxidative degradation of the polyethylene-containing mixture with an oxygen-containing reaction gas consisting of at least 5% by volume of oxygen in the presence of at least one catalyst in particular at least one catalyst which is insoluble in the reaction mixture at a process pressure of at least 1 bar, e.g. in the range from 1 to 100 bar, a reaction temperature above the melting point of the PE-containing mixture and preferably a reaction time of 0.1 to 16 h, whereby the product mixture obtained has an acid number of at least 100 mg KOH/g. As described above, the product mixture obtained is a water-insoluble mixture of a homologous series of a plurality of different α,ω-n-alkyl di-carboxylic acids with a carbon chain length of at least C₈.

According to the invention α,ω-n-alkyl dicarboxylic acids are understood to be saturated carboxylic acids with two carboxylic acid groups, wherein the linear alkyl chain is substituted at position 1 (α-position) and at the terminal position (ω-position) with a carboxylic acid group. Water-insoluble α,ω-n-alkyl dicarboxylic acids with a carbon chain length of at least C₈ are obtained by the process according to the invention. Water-insoluble is understood to mean that the α,ω-n-alkyl dicarboxylic acids have a solubility (at 20° C.) of less than 3 g/L. For example azelaic acid (C₉) has a water solubility of approx. 2.4 g/L at 20° C. Due to secondary effects, cork acid (C₈) has an even slightly lower solubility, whereby the water solubility generally decreases with increasing chain length. Accordingly, according to the invention a compound in particular a hydrocarbon-based compound with a water solubility of 3 g/L or more at 20° C. is understood to be water-soluble.

According to the invention a mixture of a homologous series of a plurality of different α,ω-n-alkyl dicarboxylic acids having a carbon chain length of at least C₈ comprises a mixture of substances or a composition of at least 3 different α,ω-n-alkyl dicarboxylic acids each having a carbon chain length of C₈ or more and wherein the carbon chains of the respective different α,ω-n-alkyl dicarboxylic acids differ by only a CH₂ group. For example in a mixture of a homologous series of a plurality of different α,ω-n-alkyl dicarboxylic acids cork acid (C₈), azelaic acid (C₉) and sebacic acid (C₁₀) are present without being limited to these C₈, C₉ and C₁₀ α,ω-n-alkyl dicarboxylic acids. Preferably, the mixture according to the invention comprises at least 5 different α,ω-n-alkyl dicarboxylic acids of a homologous series, more preferably at least 10 different α,ω-n-alkyl dicarboxylic acids of a homologous series, each having a carbon chain length of C₈ or more.

Preferably, the α,ω-n-alkyl dicarboxylic acids defined above are C₈-C₃₄-α,ω-n-alkyl di-carboxylic acids, more preferably C₈-C₂₆-α,ω-n-alkyl dicarboxylic acids and particularly preferably C₉-C₁₈-α,ω-n-alkyl dicarboxylic acids. This means that the process according to the invention can preferably be used to obtain mixtures of linear C₈-C₃₄-α,ω-alkyldicarboxylic acids, more preferably C₈-C₂₆-α,ω-alkyldicarboxylic acids and particularly preferably C₉-C₁₅-α,ω-alkyldicarboxylic acids. Consequently, the composition according to the invention preferably consists essentially of a mixture of a homologous series of at least 10 different linear α,ω-alkyl di-carboxylic acids with a carbon chain length in the range from C₈ to C₂₆, more preferably C₉ to C₁₈.

The method according to the invention comprises providing a (starting) mixture (plastics mixture) comprising at least 50% by mass of polyethylene (PE), in particular at least 70% by mass of PE, e.g. 50 to 100 mass-% PE or 70 to 100 mass-% PE, wherein the PE comprises 5.0 mass %, in particular 20 mass-% or more high-density polyethylene (HDPE) and wherein the polyethylenes contained have a weight-average molecular weight (Mw) of 20,000 g/mol or higher as is typical for melt-processed plastic waste.

According to the invention the PE-containing mixture is not further limited provided that it contains at least 50% by mass PE, in particular at least 70% by mass PE, which in turn contains 5.0% by mass or more, in particular at least 20% by mass HDPE with an Mw of 20,000 g/mol or higher. The mixture defined above is generally a heterogeneous or homogeneous mixture of substances. Preferably, it is a homogeneous or heterogeneous granulate, films, hollow bodies, composite materials, production waste or other melt-processed bodies. The mixture can also be for example a solution, a suspension, an emulsion, a homogeneous or heterogeneous powder.

Preferably, the plastic mixture is a secondary raw material stream as it occurs in the German and European plastic waste management industry and is defined and also traded for example by various national standards (e.g. green dot in Germany). The plastic mixture employed can be completely i.e. 100% by mass a secondary raw material. However, it is also possible to employ secondary raw materials together with freshly synthesized PE and/or HDPE. Preferably, the PE-containing mixture employed has a secondary raw material content of at least 50% by mass. According to the present invention secondary raw materials are understood to be all PE-containing materials that have undergone melt processing. In addition to recyclates, this also includes offcuts. However, the present invention is not limited to the use of PE-containing secondary raw materials and pure, i.e. freshly synthesized and unprocessed, polyethylenes and in particular HDPE can also be employed.

According to the invention the plastic mixture containing foreign substances comprises 50.0% by mass or more of polyethylenes, preferably 70.0% by mass or more of polyethylenes, more preferably 90.0% by mass or more of polyethylenes and particularly preferably 95.0% by mass or more of polyethylenes. The upper % by mass limit for the polyethylenes contained in the plastic mixture is not further limited according to the invention this upper % by mass limit preferably being 100.0% by mass or less, more preferably 80.0% by mass or less and particularly preferably 70.0% by mass or less. The substance mixtures defined above can be for example 50.0 to 100.0% by mass, 50.0 to 90.0% by mass, 50.0 to 80.0% by mass, 50.0 to 70.0% by mass, 60.0 to 100, 0% by mass, 60.0 to 90.0% by mass, 60.0 to 80.0% by mass, 60.0 to 70.0% by mass, 70.0 to 100.0% by mass, 70.0 to 90.0% by mass, or 70.0 to 80.0% by mass of polyethylenes.

According to the invention, the PE present in the mixture contains 5.0% by mass or more of HDPE, preferably 20.0% by mass or more of HDPE, more preferably 40.0% by mass or more of HDPE and particularly preferably 60.0% by mass or more of HDPE. The upper % by mass limit for the HDPE contained in the PE is not further limited according to the invention, this upper % by mass limit preferably being 90.0% by mass or less, more preferably 80.0% by mass or less and particularly preferably 70.0% by mass or less. The PE contained in the mixture defined above can be, for example, 5.0 to 90.0% by mass, 5.0 to 80.0% by mass, 5.0 to 70.0% by mass, 20.0 to 90.0% by mass, 20.0 to 80.0% by mass, 20.0 to 70.0% by mass, 40.0 to 90.0% by mass, 40.0 to 80.0% by mass, 40.0 to 70.0% by mass, 60.0 to 90.0% by mass, 60.0 to 80.0% by mass or 60.0 to 70.0% by mass HDPE.

According to a preferred embodiment a PE-containing mixture is used which has a secondary raw material content of at least 50% by mass. It is particularly preferred if the polyethylene-containing secondary raw material stream employed has a total polyethylene content of at least 90% by mass and a relative HDPE content of at least 50% by mass.

According to the invention, the HDPE contained in the PE defined above and the polyethylenes contained in the plastic mixture have a weight-average molecular weight (Mw) of 20,000 g/mol or higher, preferably 30,000 g/mol or higher, more preferably 40,000 g/mol or higher and particularly preferably 50,000 g/mol or higher. According to the invention, the upper limit for the Mw of the HDPE contained in the PE and of the polyethylenes is not further limited.

The HDPE contained in the PE defined above preferably has a crystallinity (also referred to as degree of crystallization or degree of crystallinity) of 50 to 80%, more preferably of 52 to 78% and particularly preferably of 54 to 76%. The crystallinity of the HDPE contained in the PE defined above can be for example 50 to 78%, 50 to 76%, 52 to 80%, 52 to 76%, 54 to 80% or 54 to 78%.

The HDPE contained in the PE defined above preferably has a density of 0.935 to 0.980 g/cm³, more preferably of 0.940 to 0.975 g/cm³ and particularly preferably of 0.945 to 0.970 g/cm³. For example, the HDPE contained in the PE defined above has a density of from 0.935 to 0.975 g/cm³, from 0.935 to 0.970 g/cm³, from 0.940 to 0.980 g/cm³, from 0.940 to 0.970 g/cm³, from 0.945 to 0.980 g/cm³ or from 0.945 to 0.975 g/cm³.

According to the invention, the HDPE contained in the PE defined above preferably has a melting temperature (Tₘ) of 120 to 145° C., more preferably of 122 to 143° C. and particularly preferably of 125 to 140° C. For example, the HDPE contained in the PE defined above has a melting temperature (Tₘ) of 120 to 143° C., of 120 to 140° C., of 122 to 145° C., of 122 to 140° C., of 125 to 145° C. or of 125 to 143° C.

The melting temperature (Tₘ) (hereinafter also referred to as melting point) of the HDPE used and of the PE-containing mixture itself is determined by dynamic differential scanning calorimetry (DSC).

In a preferred embodiment of the process according to the invention the PE contained in the plastic mixture further contains low-density polyethylene (LDPE) and/or linear low-density polyethylene (LLDPE).

According to the invention the content of LDPE and/or LLDPE in the plastics mixture defined above is not further limited as long as the mixture according to the invention has a relative HDPE content of 5.0% by mass or more. The content of LDPE and/or LLDPE in the PE defined above may be, for example, 95.0% by mass or less, 90.0% by mass or less, 85.0% by mass or less, 80.0% by mass or less, 75.0% by mass or less, 70.0% by mass or less, 65.0% by mass or less, 60.0% by mass or less, 55, 0% by mass or less, 50.0% by mass or less, 45.0% by mass or less, 40.0% by mass or less, 35.0% by mass or less, 30.0% by mass or less, 25.0% by mass or less, 20.0% by mass or less, 15.0% by mass or less, 10.0% by mass or less, 5.0% by mass or less or 0% by mass.

The crystallinity of the LDPE defined above is preferably from 40 to less than 50%, more preferably from 41 to 49% and particularly preferably from 42 to 48%. The crystallinity of the LDPE defined above can be, for example, from 40 to 49%, from 40 to 48%, from 41 to less than 50%, from 41 to 48%, from 42 to less than 50% or from 42 to 49%.

The LDPE defined above preferably has a density of 0.915 to less than 0.935 g/cm³ and more preferably of 0.920 to 0.930 g/cm³. For example, the LDPE defined above has a density of from 0.915 to 0.930 g/cm³ or from 0.920 to less than 0.935 g/cm³.

The LDPE defined above preferably has a melting temperature (Tₘ) of 100 to 135° C., more preferably 105 to 130° C. and particularly preferably 110 to 124° C. For example, the LDPE defined above has a melting temperature (Tₘ) of 100 to 130° C., of 100 to 124° C., of 105 to 135° C., of 105 to 124° C., of 110 to 135° C. or of 110 to 130° C.

The crystallinity of the LLDPE defined above is preferably from 10 to less than 50%, more preferably from 15 to 45% and particularly preferably from 20 to 40%. The crystallinity of the LLDPE defined above can be, for example, from 10 to 45%, from 10 to 40%, from 15 to less than 50%, from 15 to 40%, from 20 to less than 50% or from 20 to 45%.

The LLDPE defined above preferably has a density of from 0.870 to less than 0.935 g/cm³, more preferably from 0.875 to 0.930 g/cm³ and particularly preferably from 0.880 to 0.925 g/cm³. For example, the LLDPE defined above has a density of from 0.870 to 0.930 g/cm³, from 0.870 to 0.925 g/cm³, from 0.875 to less than 0.935 g/cm³, from 0.875 to 0.925 g/cm³, from 0.880 to less than 0.935 g/cm³ or from 0.880 to 0.930 g/cm³.

The LLDPE defined above preferably has a melting temperature (Tₘ) of 45 to 135° C., more preferably 75 to 130° C. and particularly preferably 110 to 124° C. For example, the LLDPE defined above has a melting temperature (Tₘ) of 45 to 130° C., of 45 to 124° C., of 75 to 135° C., of 75 to 124° C., of 110 to 135° C. or of 110 to 130° C.

Typical methods for determination of the crystallinity of a polymer, in particular HDPE, LDPE and LLDPE as defined above are known to the skilled person. The crystallinity of HDPE, LDPE and LLDPE as defined above can be determined for example by dynamic differential scanning calorimetry (DSC), X-ray diffraction, IR spectroscopy or NMR spectroscopy. Preferably, the crystallinity is determined using dynamic differential scanning calorimetry (DSC).

Typical methods for determining the M_{W} of a polymer in particular of HDPE, LDPE or LLDPE are known to the skilled person. The M_{W} of HDPE, LDPE or LLDPE can be determined for example by gel permeation chromatography (GPC), analytical ultracentrifugation (AUC), matrix-assisted laser desorption ionization time-of-flight mass spectrometry (MALDI-ToF-MS), electrospray ionization time-of-flight mass spectrometry (ESI-ToF-MS), or asymmetric flow field-flow fractionation (AF4). Preferably, the determination is carried out via gel permeation chromatography (GPC) using polystyrene standards and universal calibration or using HDPE standards and linear calibration, whereby a modified polystyrene cross-linked with divinylbenzene can be used as column material. The GPC measurements can be carried out at 160° C. with di- or trichlorobenzene as solvent.

In the process according to the invention the oxidative degradation of the polyethylene-containing mixture is carried out with an oxygen-containing reaction gas in the presence of at least one catalyst. The catalyst is preferably added to the PE-containing mixture.

The catalyst may for example be a single catalyst or a combination of two or more catalysts, e.g. 2 or 3 catalysts. In this case the catalysts are different e.g. with regard to their solubility in the reaction mixture and/or with regard to their chemical constitution. According to the invention, the catalysts can be selected from the group of organic catalysts, such as N-hydroxyphthalimide (NHPI) and from inorganic catalysts, such as metals and metal compounds in particular noble metals and metal compounds. The metals are preferably selected from the group of Mo, Rh, Pd, Ag, W, Re, Os, Ir, Pt and Au. In addition to these noble metal catalysts, metal salts can preferably also be used as catalysts. The cation of the metal salts is preferably selected from the group of Mn, Fe, Co, Nb, Ni, Cu, Zn, Cr, V, Ti, Ru, Rh, Pd, Ag, Mo, W, Re, Os, Ir, Pt and Au. The metals and metal compounds (metal salts) can be described as follows: Mn(X)_{2/4/6}, Fe(X)_{2/3}, Co(X)_{2/3}, Ni(X)_{2/3}, Nb(X)₅, Cu(X)_{1/2}, Zn(X)₂, Cr(X)_{2/3/6}, V(X)_{2/3/4/5}, Ti(X)_{2/4}, Ru(X)_{2/3/6}, Rh(X)_{0/1/2/3/4}, Pd(X)_{0/2/4}, Ag(X)_{0/1/2}, Au(X)_{0/1/2/3}, W(X)_{0/2/3/4/5/6}, Mo(X)_{0/2/3/4/5/6}, Re(X)_{0/4/7}, Os(X)_{0/4/8}, Ir(X)_{0/4/8}, or Pt(X)_{0/2/4/6}.

According to the invention the anion/anions of the metal salts defined as X is/are not further limited provided that a salt is present in combination with metal ions. The anion/anions of the metal salts can be for example at least one laurate, at least one myristate, at least one palmitate, at least one palmitoleate, at least one stearate, at least one oleate, at least one erucate, at least one naphthenate, at least one acetate, at least one acetylacetonate, at least one chloride, at least one bromide, at least one iodide, at least one nitrate, at least one oxide, at least one sulfate, at least one carbonate or at least one phosphate.

Preferred catalysts are transition metal compounds, in particular transition metal compounds from the group consisting of cobalt, nickel, manganese, niobium, chromium, iron, copper, zinc, ruthenium and vanadium. In particular, the catalyst comprises at least one compound of manganese, in particular a manganese oxide and specifically manganese(IV) oxide, which can be used alone or in combination with at least one other of the aforementioned catalysts. With these catalysts, in particular the Mn-containing catalysts, good conversions and good selectivities are achieved with regard to the formation of α,ω-alkyl dicarboxylic acids with a carbon chain length of at least C₈.

Likewise preferably the catalyst comprises N-hydroxyphthalimide, which can be used alone or in combination with at least one other of the aforementioned catalysts.

Preferably, the catalyst comprises at least one catalyst which is not soluble in the reaction product. A catalyst which is not soluble in the reaction product is understood to be a catalyst which is not soluble or only soluble to a very limited extent in the product mixture at 100° C. and 1 bar. Insoluble or very limited solubility means that the solubility of the catalyst in the product mixture at 100° C. and 1 bar is less than 0.01 g of catalyst per kg of product mixture.

The solubility of the catalyst can be determined, for example, by adding the catalyst to a melt of the product mixture in an amount of 100 g/kg of the product mixture, holding the product mixture at 100° C. for 1 h, separating the undissolved catalyst from the melt at 100° C. and then determining the content of the catalyst in the mixture, for example by energy dispersive X-ray spectrometry (EDX) or by inductively coupled plasma mass spectrometry (ICP-MS).

Since the product mixture essentially consists of linear α,ω-alkyl dicarboxylic acids with a carbon chain length of at least C₈ and corresponding keto-functionalized and/or hydroxy-functionalized α,ω-alkyl dicarboxylic acids, the exact composition is negligible for the solubility.

Surprisingly, a limited solubility or insolubility of the catalyst in the reaction mixture is not detrimental to the efficiency of oxidative degradation and it is therefore possible with these catalysts to achieve good conversions and good selectivities with respect to the formation of α,ω-alkyl dicarboxylic acids with a carbon chain length of at least C₈. At the same time the low solubility of the catalyst facilitates its separation from the reaction mixture and thus the recovery of the catalyst.

Examples of catalysts that are not soluble in the reaction mixture are in particular all transition metal oxides, noble metals, supported noble metals and supported metal salts which can be used alone or in combination. Examples of suitable transition metal oxides are, in particular, manganese oxides, e.g. manganese(IV) oxide (MnO₂), manganese(III)oxide, vanadium oxides such as vanadium(V)oxide, niobium oxides, e.g. niobium(V)oxide, copper oxides, in particular CuO and Cu₂O, silver oxides, in particular Ag₂O, zinc oxides, in particular ZnO, iron oxides, in particular FeO, Fe₂O₃ and Fe₃O₄, cobalt oxides, in particular CoO, Co₂O₃ and Co₃O₄, chromium oxides of oxidation states III and IV (Cr₂O₃ and CrO₂), ruthenium oxides of oxidation states III and IV (Ru₂O₃ and RuO₂) and nickel oxides, e.g. NiO, Ni₂O₃. Particularly suitable precious metals are palladium or platinum, which can be used as such or in supported form, e.g. on carrier materials such as activated carbon, silicon dioxide, zeolites, aluminum oxide, magnesium chloride or barium sulfate.

In a preferred embodiment of the present invention, a binary catalyst system is preferably employed. The selection of the respective catalysts must be adapted to the PE-containing mixtures to be used and the process conditions. For example, Co and Mn salts are preferably used in combination without being limited to this. Ni salts can also preferably be combined with a further metal compound as defined above. Combinations of at least one manganese salt and N-hydroxyphthalimide are also preferred.

Furthermore, it has been found to be advantageous to employ a catalyst mixture comprising at least two catalysts, at least one of the catalysts comprising at least one catalyst which is not soluble in the reaction product and at least one catalyst which is soluble in the reaction mixture. This allows to retain the advantages of easy separation of the catalyst which is not soluble in the reaction product and to compensate for its sometimes somewhat lower activities in the induction phase by the presence of the catalyst which is soluble in the reaction mixture. Preferably, the catalyst which is not soluble in the reaction product is used in at least the same or greater quantity than the catalyst which is soluble in the reaction mixture e.g. in a mass ratio of 20:1 to 1:1.

Catalysts which are not soluble in the reaction product are primarily the above-mentioned transition metal oxides, noble metals, supported noble metals and supported metal salts, which can be used alone or in combination.

Catalysts which are soluble in the reaction product are understood to be catalysts which have a solubility of at least 0.01 g/kg, in particular at least 0.1 g/kg, especially at least 1 g/kg, in the product mixture at 100° C. and 1 bar.

The catalysts soluble in the reaction product include in particular certain transition metal salts, in particular salts of cobalt and manganese, in particular their salts with C₂-C₂₀ alkane carboxylic acids, for example their acetates, propionates, butyrates, caprylates, decanoates, dodecanoates, myristates, palmitates and stearates, their salts or complexes with 1,3-diketones, e.g. their acetylacetonates, and in particular N-hydroxyphthalimide.

Preferably, the catalyst or the catalyst mixture is used in a total amount of at least 0.0001% by mass, more preferably at least 0.001% by mass, even more preferably at least 0.005% by mass, especially at least 0.01% by mass, based on 100% by mass of the PE-containing mixture defined above e.g. in a total amount of 0.0001% by mass, more preferably at least 0.005% by mass, especially at least 0.01% by mass, based on 100% by mass of the PE-containing mixture defined above. For example in a total amount of 0.0001 to 10.0% by mass, more preferably 0.001 to 5.0% by mass and particularly preferably 0.005 to 3.0% by mass. The PE-containing mixture defined above may for example contain the catalyst or the catalyst mixture in a total amount of preferably 0.0001 to 2.0% by mass, 0.0001 to 1.0% by mass, 0.001 to 3.0% by mass, 0.001 to 2.0% by mass, 0.001 to 1.0% by mass, 0.01 to 3.0% by mass, 0.01 to 2.0% by mass or 0.01 to 1.0% by mass.

Since the PE-containing mixture used according to the invention contains at least one of the catalysts defined above, the yield of oxidative cleavage products in a given time frame, the selectivity with regard to the formation of the desired linear α,ω-alkyl dicarboxylic acids with a carbon chain length of at least C₈ and the acid number obtained can be influenced in an advantageous manner. The catalysts thus have an influence on the chemoselectivity as well as the velocity of the oxidation reactions taking place under given physical conditions. The use of the above-mentioned catalysts thus makes it possible to carry out the reaction economically at different physical parameters, which in turn is decisive for controlling the chain length of the α,ω-n-alkyl dicarboxylic acids obtained.

In the process according to the invention, keto-functionalized and/or hydroxy-functionalized α,ω-n-alkyl dicarboxylic acids can also be obtained as by-products.

The functionalization of the polyethylene chain by keto groups and/or hydroxy groups represents an intermediate step before oxidative cleavage to linear α,ω-alkyl dicarboxylic acids. By using at least one of the catalysts defined above, the content of keto-functionalized and/or hydroxy-functionalized α,ω-alkyl dicarboxylic acids can be controlled by influencing the ratio of chain functionalization by keto groups and/or hydroxy groups and subsequent chain cleavage of these same groups.

Thus, the described process can be used flexibly for a broad spectrum of accessible linear α,ω-alkyl dicarboxylic acids of different chain lengths and also keto-functionalization or hydroxy-functionalization.

In a further preferred embodiment of the process according to the invention, the above-defined mixture further comprises at least one aqueous reaction medium.

The aqueous reaction medium is a medium comprising at least water, wherein the water may be, for example, drinking water or distilled water.

The above reaction medium may contain one or more inorganic bases, such as NaOH and/or KOH. However, the reaction medium may also contain or consist of an organic acid.

It is also possible for the reaction medium to (additionally) contain or consist of an organic solvent. The organic acid can be, for example, acetic acid, propionic acid, butyric acid, myristic acid, palmitic acid, stearic acid, oleic acid or benzoic acid. The organic solvent can be for example benzonitrile or acetonitrile.

Particularly preferably, the aqueous reaction medium consists of water.

If the aqueous reaction medium consists of water and is contained in the mixture defined above, the process according to the invention (a) uses an environmentally friendly reaction medium and (b) the mixture can be purified directly following the heating according to the invention described below for example by means of steam distillation, which in turn leads to an increase in efficiency of the process according to the invention. The reason for this lies in the fact that the purification of the reaction mixture can be carried out directly from the reaction medium.

In addition, undesirable water-soluble by-products and impurities can be separated simultaneously.

The mixture defined above preferably contains 0.1 to 100.0 mass equivalents, more preferably 1 to 50.0 mass equivalents and particularly preferably 2.5 to 10.0 mass equivalents of the aqueous reaction medium defined above in relation to 1 mass equivalent of the plastic mixture defined above.

The mixture defined above can be, for example, 0.1 to 50.0 mass equivalents, 0.1 to 10.0 mass equivalents, 1 to 100.0 mass equivalents, 1 to 50.0 mass equivalents, 1 to 10.0 mass equivalents, 2.5 to 100, 0 mass equivalents, 2.5 to 50.0 mass equivalents, 2.5 to 10.0 mass equivalents of the aqueous reaction medium defined above in relation to 1 mass equivalent of the plastic mixture defined above.

However, the process according to the invention does not have to be carried out in an aqueous reaction medium. It is also preferable to carry out the oxidative degradation of the PE-containing mixtures in the polymer melt.

The above mixture may contain further additives and foreign substances according to the invention provided that these do not prevent the oxidative degradation of polyethylene with oxygen according to the invention. Such further additives and foreign substances may be for example liquid and solid plasticizers, stabilizers, lubricants and mould release agents, compatibilizers, nucleating agents, fibrous or non-fibrous fillers, dyes, solvent residues, food residues, general product residues (e.g. shampoo), glass, ceramics, rubber, stones, wood, textiles, paper, cardboard, paperboard, metal foils, polyether, polyester, polyamides, polyurethanes, polycarbonates, PP, PS, PVC or combinations thereof.

Advantageously, initiators can be added to the mixture instead of or in addition to the catalysts before or while the mixture is heated to the desired temperature. By using initiators such as ketones or radical initiators the oxidative degradation of the PE-containing mixtures can be accelerated. Initiators known in the prior art can be used for this purpose.

In contrast to the processes known in the prior art for the oxidative degradation of polyolefins no strong oxidizing agents such as nitric acid, ozone or peroxides are used in the process according to the invention.

The process according to the invention further comprises heating the prepared mixture at a temperature above the melting point of the plastics contained in the starting mixture and 300° C. or less and a pressure of 1 to 100 bar of an oxygen-containing reaction gas for at least 0.1 h, e.g. 0.1 to 16 h, wherein the oxygen-containing reaction gas contains 5% by volume or more oxygen.

According to the invention, the heating of the above-defined mixture is not further limited provided that the mixture is heated to a temperature above the melting point of the PE-containing mixture and a pressure of 1 to 100 bar of the above-defined oxygen-containing reaction gas for at least 0.1 h, e.g. 0.1 to 16 h. The mixture can be heated for example by means of a heating block, heating jacket or supply of a preheated gas stream.

The heating of the mixture as defined above can for example be carried out in a reactor. According to the invention the reactor is not further limited provided hat a chemical reaction of the mixture defined above can be carried out in it under the specified process conditions. The reactor can for example be a V2A stainless steel autoclave or a general high-pressure reactor made of a similarly corrosion-resistant material.

According to the invention the mixture defined above is heated to a temperature above the melting point of the PE-containing mixture. The melting point of the PE-containing mixture depends on the chemical composition of the plastic mixture used. For example the plastic mixture can be heated to a temperature of 140 to 300° C. if the melting point is less than 140° C. Preferably, the heating step is carried out at a temperature of 143 to 250° C., more preferably at a temperature of 147 to 220° C. and particularly preferably at a temperature of 150 to 200° C. For example, the mixture according to the invention can be heated at a temperature of 140 to 250° C., 140 to 220° C., 140 to 200° C., 143 to 300° C., 143 to 250° C., 143 to 220° C., 143 to 200° C., 147 to 300° C., 147 to 250° C., 147 to 220° C., 147 to 200° C., 150 to 300° C., 150 to 250° C., 150 to 220° C. or 150 to 200° C.

According to the invention, the mixture defined above is heated at a pressure of 1 to 100 bar of the oxygen-containing reaction gas, preferably at a pressure of 5 to 70 bar, more preferably at a pressure of 10 to 50 bar and particularly preferably at a pressure of 20 to 30 bar. The mixture according to the invention can for example be heated at a pressure of 1 to 70 bar, 1 to 50 bar, 1 to 30 bar, 5 to 100 bar, 5 to 70 bar, 5 to 50 bar, 5 to 30 bar, 10 to 100 bar, 10 to 70 bar, 10 to 50 bar, 10 to 30 bar, 20 to 100 bar, 20 to 70 bar, 20 to 50 bar or 20 to 30 bar of the oxygen-containing reaction gas.

The oxygen-containing reaction gas according to the invention is not further limited provided that the gas mixture contains 5.0% by volume or more of oxygen. The oxygen-containing reaction gas defined above preferably contains 10.0% by volume or more, more preferably 15.0% by volume or more and particularly preferably 20.0% by volume or more of oxygen. According to the invention the upper limit in % by volume of oxygen in the oxygen-containing reaction gas defined above is not further limited. The oxygen-containing reaction gas defined above may in particular be air, synthetic air (20.0% by volume of oxygen and 80.0% by volume of nitrogen) or 100.0% by volume oxygen. Preferably, the oxygen-containing reaction gas contains less than 1% by volume of nitrous gases such as NO and NO₂.

The oxygen-containing reaction gas is particularly preferably atmospheric air, which increases the cost efficiency, sustainability and environmental friendliness of the process according to the invention.

According to the invention, the above-defined mixture is heated for at least 0.1 h, in particular at least 0.5 h and especially at least 1 h, e.g. 0.1 to 16 h, preferably 0.5 to 8 h, more preferably 1 to 4 h and particularly preferably 1 to 3 h.

In a preferred embodiment of the process according to the invention, the prepared mixture is heated at a temperature of 150 to 200° C. and an air pressure of 20 to 30 bar for 1 to 4 hours.

According to the invention a plurality of different linear α,ω-alkyl dicarboxylic acids with a carbon chain length of at least C₈ is obtained by oxidative degradation of the PE-containing mixtures used. According to a preferred embodiment of the present invention at least 50 mol % of the detectable products obtained in the product mixture have a carbon chain length in the range of C₈ to C₃₄. Particularly preferred mixtures of linear C₈-C₂₆-α,ω-alkyl dicarboxylic acids and in particular C₉-C₁₅-α,ω-alkyl dicarboxylic acids are obtained using the process according to the invention. The chain length distribution of the α,ω-alkyl dicarboxylic acids in the resulting product mixture can be determined by gas chromatography using linear α,ω-alkyl di-carboxylic acids as reference substances.

According to the invention the maximum and the width of the detectable chain length distribution of the product mixture can be adjusted by varying the physical reaction conditions during the process and/or the catalyst employed. In particular, the average carbon chain length of the product mixture and its dispersity can be selectively adjusted by varying these reaction conditions. For example by varying the reaction temperature and/or the presence of at least one catalyst during the process according to the invention the distribution of the linear α,ω-alkyl di-carboxylic acids in the product mixture can be selectively adjusted. Such a variation of the reaction conditions can therefore be regarded as a two-stage or multi-stage process. Without being limited to this the process according to the invention can for example be carried out as a two-stage process, in which first the oxidation of the polyethylenes takes place at higher temperatures and/or pressures and then, in a second step, the chain length distribution is adjusted under milder conditions, possibly in the presence of the at least one catalyst. These steps do not necessarily have to take place spatially separated and can differ, for example, only by a different temperature and/or pressure and/or by the use of a catalyst or a different catalyst in the same reactor. It is also possible to control the product mixture by varying the reaction medium. For example, the process according to the invention can initially be carried out in the polymer melt, i.e. without an aqueous reaction medium and can be continued as an aqueous reaction in a further step.

In a further preferred embodiment, the process according to the invention further comprises: optionally cooling the heated mixture depending on the subsequent step; and purifying the (cooled) mixture by at least one selected from the group consisting of filtration, drying, precipitation, extraction, centrifugation, crystallization, fractional distillation, sublimation, steam distillation and column chromatography.

According to the invention cooling of the heated mixture as defined above is not further limited provided that the heated mixture cools down in the process. Preferably, the heated mixture as defined above is cooled down to a temperature of 20 to 99° C.

If a catalyst is used in the process according to the invention which comprises at least one catalyst which is not soluble in the reaction product this catalyst can be separated from the reaction mixture or the reaction product after the reaction with the oxygen-containing reaction gas. Separation can be achieved for example by emulsifying the resulting reaction product in an aqueous base e.g. aqueous alkali when reacting in the melt or by making the resulting emulsion alkaline by adding a base for example by adding alkali metal hydroxides when reacting the PE-containing mixture in an aqueous suspension or emulsion. Thereby the product mixture or the α,ω-alkyl dicarboxylic acids and their keto- or hydroxy-functionalized derivatives contained therein dissolve. Preferably, the base or alkali metal hydroxide is used in such a way that complete neutralization of the carboxyl groups contained in the product mixture is achieved. In particular, the base is used in over-stoichiometric quantities. The catalyst which is usually insoluble not only in the product mixture but also in water can then be easily separated by solid-liquid separation such as filtration, centrifugation or decantation. Similarly, the organic product mixture can be dissolved in an organic solvent in which the catalyst is usually also insoluble and then the catalyst can be separated from the solution by means of one of the above-mentioned solid-liquid separation measures.

In this way product mixtures can be obtained which contain less than 150 ppm of the catalyst based on the product mass and in the case of metal-containing catalysts exhibit a catalyst metal content of less than 100 ppm based on the product mass.

Purification processes such as filtration, drying, precipitation, extraction, centrifugation, crystallization, fractional distillation, sublimation, steam distillation and column chromatography are known to the skilled person and can be carried out in combination one after the other or individually.

For example, the (cooled) mixture defined above can be dissolved in an organic solvent such as xylene, precipitated by adding the dissolved mixture to a further solvent, such as methanol, and then either filtered or centrifuged and then dried. Preferably, the mixture can be purified by aqueous extraction with for example a sodium hydroxide solution and the soluble fraction can be obtained as a solid by precipitation in a neutralization bath. This can then be filtered or centrifuged and dried.

Alternatively, the (cooled) mixture defined above, if it contains an organic solvent such as benzonitrile, can also be extracted directly with a basic aqueous solution such as a sodium hydroxide solution. The soluble fraction can be obtained as a solid by precipitation in a neutralization bath. This can then be filtered or centrifuged and dried.

For example, the (cooled) mixture defined above can also be purified by steam distillation if it also contains water or if water is added. For this purpose, the (cooled) mixture can either be reheated or the resulting water vapor containing the purified mixture can be removed directly in the reaction heat and cooled separately so that the purified mixture precipitates as a solid and can then be filtered and dried. For example, the (cooled) mixture defined above can be purified directly using fractional distillation or acid chromatography.

After the reaction time has elapsed the heated mixture defined above can be purified directly by fractional distillation or steam distillation without having to cool the heated mixture beforehand. Therefore, in a further preferred embodiment, the process according to the invention further comprises: purifying the heated mixture by fractional distillation or by steam distillation provided that the heated mixture already contains water.

The product mixture optionally obtained by means of the purification processes described above has an acid value of at least 100 mg KOH/g. In a preferred embodiment of the process according to the invention, the optionally purified mixture has an acid number of at least 150 mg KOH/g and particularly preferably of at least 200 mg KOH/g. For example, 600 mg KOH/g, preferably 500 mg KOH/g, particularly preferably 400 mg KOH/g can be mentioned as the upper limit. The acid number of the resulting mixture can be determined for example by titration as described in the following examples.

In a further preferred embodiment of the present invention, the product mixture comprises at least 5% by mass, preferably 5 to 50% by mass, more preferably 5 to 20% by mass and particularly preferably 5 to 10% by mass of keto- and/or hydroxy-functionalized α,ω-n-alkyl dicarboxylic acids, wherein the keto- and/or hydroxy-functionalization is defined by at least one ketone group and/or hydroxy group between the two carboxylic acid groups. The mixture defined above may contain for example 5 to 20% by mass, 5 to 10% by mass, 10 to 50% by mass, 10 to 20% by mass or 20 to 50% by mass of keto- and/or hydroxy-functionalized α,ω-n-alkyl dicarboxylic acids.

Accordingly, the present invention also relates to a mixture of a homologous series of a plurality in particular of at least 4 in particular at least 5 different linear α,ω-alkyl dicarboxylic acids having a carbon chain length of at least C₈, in particular at least C₉ which is obtainable by the process according to the invention and wherein at least 50 mol %, in particular at least 70 mol % of the detectable products obtained have a carbon chain length in the range of C₈ to C₃₄ and in particular a carbon chain length in the range of C₁₂ to C₃₄ as determined by gas chromatography using linear α,ω-alkyl dicarboxylic acids as reference substances. Such products could neither be obtained by conventional syntheses of linear α,ω-alkyl dicarboxylic acids, nor by degradation reactions of polyolefins.

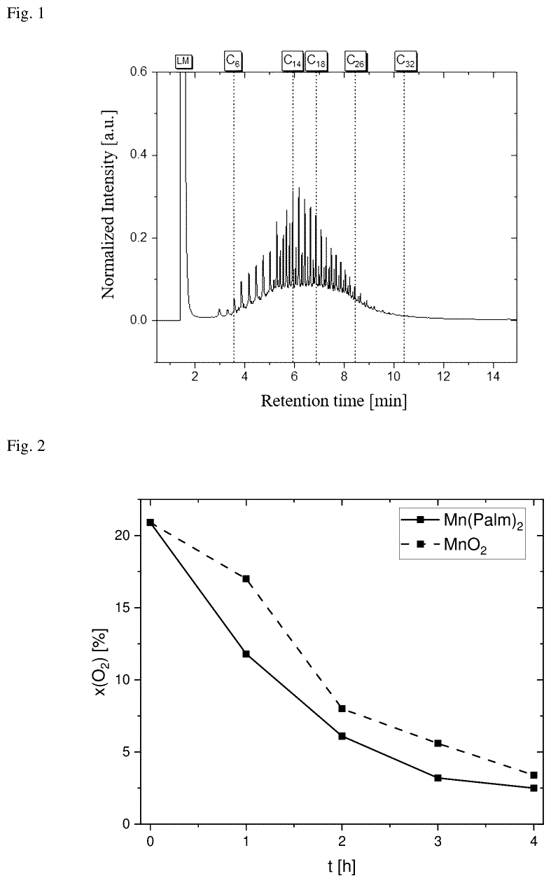

In particular, the average carbon chain length of the mixture which is defined as the maximum of a distribution determined by gas chromatography using linear α,ω-alkyl dicarboxylic acids as reference substances is ≥C₉. In particular, the maximum is in the range from C₁₂ to C₂₈ and especially in the range from C₁₃ to C₂₇. As shown in FIG. 1 the maximum of a distribution obtained by gas chromatography which is referred to as the average carbon chain length of the mixture, can be C₁₅.

In particular, mixtures or compositions of a homologous series of a plurality in particular at least 4, especially at least 5 different linear α,ω-alkyl dicarboxylic acids are obtainable by the method according to the invention which have a high proportion of linear α,ω-alkyl di-carboxylic acids with a carbon chain length of at least C₁₉. In particular, such mixtures and compositions contain at least 5% by mass, in particular at least 10% by mass e.g. 5 to 80% by mass, in particular 10 to 75% by mass of α,ω-alkyl dicarboxylic acids with a chain length ≥C₁₉. In particular, such mixtures and compositions contain at least 10% by mass, in particular 10 to 80% by mass α,ω-alkyl dicarboxylic acids with a chain length in the range of C₁₉ to C₃₄.

In particular, mixtures or compositions of a homologous series of a plurality in particular at least 4, especially at least 5 different linear α,ω-alkyl dicarboxylic acids are obtainable by the process according to the invention which mixtures or compositions comprise at least 5% by mass, in particular at least 10% by mass e.g. 5 to 80% by mass, in particular 10 to 75% by mass of α,ω-alkyl dicarboxylic acids with a chain length in the range from C₁₉ to C₃₄ and whose average carbon chain length determined by gas chromatography according to the method explained above is in the range from C₁₂ to C₂₈ and in particular in the range from C₁₃ to C₂₇.

In particular, mixtures or compositions of a homologous series of a plurality, in particular at least 4, especially at least 5 different linear α,ω-alkyl dicarboxylic acids are obtainable by the process according to the invention, which comprise at least 5% by mass, in particular at least 10% by mass, e.g. 5 to 80% by mass, in particular 10 to 75% by mass of α,ω-alkyl di-carboxylic acids with a chain length in the range C₁₉ to C₃₄ and their average carbon chain length, determined by gas chromatography according to the method explained above is in the range from C₁₂ to C₂₈ and especially in the range from C₁₃ to C₂₇ and which contains at least 5% by mass, preferably 5 to 50% by mass, more preferably 5 to 20% by mass and particularly preferably 5 to 10% by mass of keto- and/or hydroxy-functionalized α,ω-n-alkyl dicarboxylic acids.

As can be further seen in FIG. 1 the resulting mixture in addition to the pentadecane di-carboxylic acid (C₁₅) also contains all homologs of the C₈, C₉, C₁₀, C₁₁, C₁₂, C₁₃, C₁₄, C₁₆, C₁₇, C₁₈, C₁₉, C₂₀, C₂₁, C₂₂, C₂₃, C₂₄, C₂₅, C₂₆, C₂₇, C₂₈, C₂₉ and C₃₀ dicarboxylic acids.

Advantageously, the mixture (or also called composition) according to the invention consists essentially of a mixture of different linear α,ω-alkyl dicarboxylic acids with a carbon chain length in the range from C₈ to C₃₄ wherein the proportion of water-soluble compounds such as water-soluble oxidation products and compounds with a carbon chain length of C₃₅ or more is less than 10% by mass. According to the present invention, the term “substantially” is understood to mean that the proportion of linear C₈-C₃₄-α,ω-alkyl dicarboxylic acids in the composition is 80% by mass or more, preferably 85% by mass or more. Preferably, the proportion of water-soluble compounds and compounds with a carbon chain length of C₃₅ or more is less than 5% by mass, particularly preferably less than 1% by mass.

In addition, the mixtures or compositions according to the invention are preferably free of methyl ketone groups, which typically occur in oxidative degradation mixtures.

According to a further preferred embodiment the mixtures or compositions according to the invention contain at least 5% by mass of hydroxy- and/or keto-functionalized α,ω-n-alkyl di-carboxylic acids. As described above, such di-carboxylic acids have at least one hydroxy and/or keto group between the two carbonic acid groups. Preferably, the proportion of such α,ω-n-alkyl dicarboxylic acids functionalized with hydroxy groups and/or keto groups is 5 to 20% by mass, particularly preferably 5 to 10% by mass.

The mixtures or compositions according to the invention are suitable for a wide range of applications. Thus, the mixtures or compositions according to the invention in particular those mixtures or compositions which contain α,ω-n-alkyl dicarboxylic acids functionalized with hydroxyl groups and/or keto groups are characterized by a good emulsifiability in water. Due to the amphiphilic properties of their salts they are therefore particularly suitable for the production of emulsifiers for example for detergents and cleaning agents such as laundry detergents, hand dishwashing detergents, cleaning agents and also for cosmetic applications. In particular, alkali metal salts e.g. sodium or potassium salts, ammonium salts and salts with secondary or tertiary amines can be considered as salts. Preferably, the carboxyl groups in the mixtures or compositions according to the invention are neutralized to at least 50%, in particular completely neutralized or even over-neutralized.

Not least because of their structural similarity to estolides (oligoesters of hydroxy fatty acids e.g. of hydrogenated castor oil) they are also suitable as a lubricant base for water-based lubricants e.g. cooling lubricants. Furthermore, these compositions and the α,ω-n-alkyl di-carboxylic acids contained therein are suitable for the production of polymers for example polyesters in particular aliphatic and aliphatic-aromatic polyesters, in particular of those which are compostable.

Due to their structure the mixtures or compositions according to the invention in particular those mixtures or compositions containing α,ω-n-alkyl dicarboxylic acids functionalized with hydroxyl groups and/or keto groups, should be characterized by good biodegradability within the meaning of the Detergents Regulation (Regulation (EC) 648/2004 of 31 Mar. 2004) determined according to the method EN ISO 14593: 1999 given in Annex III of Regulation (EC) 648/2004.

The following examples and the accompanying figures serve to illustrate, but are not limited to, the present invention.

FIG. 1 shows a gas chromatogram of the chain length distribution of the product of a reaction carried out analogously to Example 1 (vide infra) at 150° C., 30 bar and 16 h and worked up according to Example 1. The sample preparation for GC analysis is described in the examples. The dashed lines show the respective retention times of α,ω-alkyl dicarboxylic acid standards. The peak of the solvent is marked with “S”.

FIG. 2 shows the development of the oxygen content in the gas phase after the reaction with manganese(II) palmitate (Mn(Palm)₂) and MnO₂ for varying reaction times. Obviously, the activities of the homogeneous catalyst Mn(Palm)₂ and the heterogeneous catalyst MnO₂ are comparable. The catalyst seems to have a slight induction phase, but after that the activities approach each other.

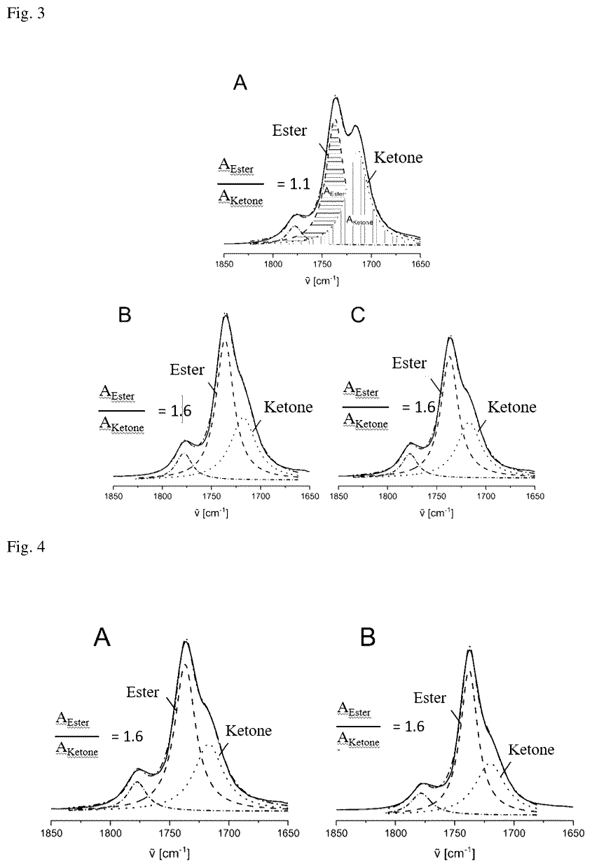

FIG. 3 shows a comparison of the infrared spectra (1650 to 1850 cm-1) of reactions A: without catalyst (example 21 below); B: with Mn(Palm)₂ (example 19 below) and C: with MnO₂ (example 15). Dashed lines show the deconvolution (Lorentz curves) of the carbonyl band into ketone and ester band. It can be seen that the homogeneous catalyst Mn(Palm)₂ and the heterogeneous catalyst MnO₂ behave essentially analogously. Without a catalyst, a higher proportion of ketone is present, which is also a disadvantage.

FIG. 4 shows a comparison of the infrared spectra (1650 to 1850 cm-1) of reactions A: with fresh MnO₂ (example 15) and B: with recovered MnO₂ (example 24). Dashed lines show the deconvolution of the carbonyl band into ketone and ester band. It can be seen that the reaction products with fresh and recycled catalyst are comparable. The same applies to the activities, as can be seen from the oxygen conversions in Table 4.

### ACID NUMBER (AN)

To determine the acid number, stock solutions were prepared gravimetrically from the respective reaction mixtures (accuracy ±0.0001 g) in i-PrOH with a concentration of approx. 10 mg/mL (±0.2). Subsequently, aliquots of 0.5 mL each corresponding to approx. 5 mg sample were taken volumetrically and again determined gravimetrically (accuracy ±0.0001 g). The total sample quantity was therefore determined completely gravimetrically. The sample was supplemented with the titration solvent to a total volume of 10 mL. The titration solvent consisted of 500 mL i-PrOH, 500 mL toluene, 1 mL deionized water and 500 mg phenolphthalein. The sample prepared in this way was titrated with freshly prepared KOH solution in i-PrOH (0.02 M) in an automated titration apparatus with optical endpoint detection. The optical endpoint was calibrated using a benzoic acid sample as a reference for each sample series and then used for the evaluation of the samples.

Carbon Chain Lengths of the Prepared α,ω-n-Alkyl Dicarboxylic Acids

The carbon chain lengths of the prepared α,ω-n-alkyl dicarboxylic acids in the product mixture to be analyzed were determined by gas chromatography (GC-FID) (Perkin Elmer Clarus 500; column length=30 m, diameter=0.320 mm, film thickness=0.25 μm, 5% phenylmethylpolysiloxane). The samples were previously treated with basic hydrazine to remove interfering ketodicarboxylic acids for GC analysis and then derivatized with N,O-bis(trimethylsilyl)trifluoroacetamide (BSTFA) as —COOTMS ester. The retention times of the different chain length ranges were defined using measurements of long-chain α,ω-n-alkyl dicarboxylic acids as standards. The respective α,ω-n-alkyl dicarboxylic acids of chain lengths C₄, C₆, C₇, C₈, C₉, C₁₀, C₁₂, C₁₄, C₁₅, C₂₃, C₂₆ and C₃₂, as shown in Table 1 below, were used for this purpose.

TABLE 1

| Chain length dicarboxylic acid reference | |
|---|---|
| (as -TMS ester) | Retention time [min] |
| 32 | 10.40 |
| 26 | 8.44 |
| 23 | 7.89 |
| 18 | 6.88 |
| 14 | 5.94 |
| 12 | 5.42 |
| 10 | 4.86 |
| 9 | 4.58 |
| 8 | 4.25 |
| 7 | 3.91 |
| 6 | 3.56 |
| 4 | 2.08 |

### Gas Phase Analysis

To determine the oxygen content of the gas phase after the reaction, the residual pressure of the steel autoclave was released into a gas sample bag. This gas sample (˜200 ml) was analyzed using an oxygen meter (ZhongAn S316).

Optical Emission Spectrometry with Inductively Coupled Plasma (ICP-OES)

The residual manganese content was determined using ICP-OES (Spektro Arcos ICP-OES) after microwave digestion with a CEM MARS6.

### Infrared Spectroscopy

20 mg of the sample obtained was esterified in a mixture of 0.5 ml chloroform, 0.5 ml methanol and 0.1 ml concentrated hydrochloric acid at 65° C. for 3 hours. The hydrochloric acid contained was neutralized by adding 100 mg sodium hydrogen carbonate and the solvent was distilled off. The resulting mixture was dissolved in 1 ml chloroform, filtered through a Teflon filter (pore size: 0.2 μm) and the solvent was distilled off. The residue was analyzed by ATR-IR spectroscopy (Perkin Elmer Spectrum 100).

The following abbreviations are used in the examples:
HDPE: high-density polyethylene
MnO₂: Manganese(IV) oxide
Mn(Palm)₂: Manganese(II) palmitate
Mn(acac)₂: Manganese(II) acetylacetonate
NHPI: N-hydroxyphthalimide
Tₘ: Melting point
DSC: Differential scanning calorimetry
M_{W}: Melting point
wt %: Percent by weight

### Example 1

First, 200 mg of granulated HDPE (Purell GB 7250, LyondellBasell Industries, density: 0.952 g/cm³, Tₘ=134° C., crystallinity=62% (DSC), MW=71.2 kg/mol) were weighed out in a glass insert and mixed with a magnetic stirring rod. This glass insert was placed in a steel autoclave (with needle valve, manometer and bursting disk). This steel autoclave was then pressurized at room temperature with synthetic air (20.0% oxygen by volume, 80.0% nitrogen by volume) to 20.0 bar, then placed in a heating block preheated to 200° C. and heated for 1 h. The reaction time represents the start of the reaction time. The start of the reaction time is represented by the insertion of the steel autoclave into the preheated heating block. After the reaction time had elapsed the steel autoclave was removed from the heating block and the pressure was immediately released to end the reaction. The melt cake was cooled to room temperature hen dissolved in 7 ml of hot xylene (130° C.) and the resulting solution precipitated in 30 ml of cold methanol (20° C.). The insoluble components were centrifuged off and the solvents (xylene and methanol) of the separated centrifugate were again removed by distillation. The resulting residue of 73 mg was then analyzed by titration and was found to have an acid value of 158 mg KOH/g product.

### Examples 2 to 5

Example 2 was carried out analogously to example 1, whereby the granulated HDPE was further mixed with 1.0% by mass manganese(II). Example 3 was prepared analogously to Example 1, with a further 1.0% by mass of iron(III) stearate being added to the granulated HDPE. Example 4 was prepared analogously to Example 1, with a further 1.0% by mass of copper(II) stearate being added to the granulated HDPE. Example 5 was prepared analogously to Example 1, with the further addition of 0.1% by mass N-hydroxyphthalimide (NHPI) and 2.5% by mass 12-tricosanone as initiator to the granulated HDPE. The results are summarized in Table 2.

### Examples 6 to 10

Examples 6 to 9 were carried out analogously to Examples 2 to 5, with the further addition of water as reaction medium to the mixture of HDPE and catalyst (according to Examples 2 to 5) (2.5 mass equivalents of water with respect to HDPE). Example 10 was carried out analogously to Example 1, whereby 0.1% by mass N-hydroxyphthalimide (NHPI) and 2.5 mass equivalents of water were added to the granulated HDPE. The results are summarized in Table 2.

### Example 11

Example 11 was performed analogously to Example 1, with the addition of 0.1% by mass manganese(II) stearate and 0.1% by mass cobalt(II) stearate to the granulated HDPE. The results are summarized in Table 2.

TABLE 2

| | Yield of centrifugate with regard | |
|---|---|---|
| Example | to HDPE employed [% by mass] | Acid number[mg KOH/g] |
| 1 | 37 | 158 |
| 2 | 48 | 187 |
| 3 | 33 | 166 |
| 4 | 26 | 159 |
| 5 | 51 | 128 |
| 6 | 49 | 144 |
| 7 | 44 | 252 |
| 8 | 28 | 181 |
| 9 | 53 | 109 |
| 10 | 38 | 146 |
| 11 | 45 | 232 |

### Example 12

200 mg of granulated HDPE (Purell GB 7250, LyondellBasell Industries, density: 0.952 g/cm³, Tₘ=134° C., crystallinity=62% (DSC), M_{W}=71.2 kg/mol) mixed with 0.77 wt % manganese (IV) oxide (MnO₂) were weighed in a glass insert. This was placed in a steel autoclave (with needle valve, manometer and bursting disk). This steel autoclave was then pressurized to 20.0 bar at room temperature using a compressor and subsequently placed in a heating block preheated to 180° C. and heated for 1 h. The start of the reaction time was represented by the insertion of the steel autoclave into the preheated heating block. After the reaction time had elapsed the steel autoclave was removed from the heating block the pressure was released and the gas phase was analyzed. The results are summarized in Tables 3 and 5.

### Examples 13 and 14

Examples 13 and 14 were performed analogously to Example 12, with reaction times of 2 h and 3 h respectively. The results are summarized in Table 3.

### Example 15

200 mg of granulated HDPE (Purell GB 7250, LyondellBasell Industries, density: 0.952 g/cm³, Tₘ=134° C., crystallinity=62% (DSC), M_{w}=71.2 kg/mol) mixed with 0.77 wt % manganese (IV) oxide was weighed in a glass insert. This was placed in a steel autoclave (with needle valve, manometer and bursting disk). This steel autoclave was then pressurized to 20.0 bar at room temperature using a compressor and then placed in a heating block preheated to 180° C. and heated for 4 hours. The start of the reaction time was represented by the insertion of the steel autoclave into the preheated heating block. After the reaction time had elapsed the steel autoclave was removed from the heating block the pressure was released and the gas phase was analyzed. (The results of this analysis are summarized in Tables 3, 4 and 6). The melt cake was cooled to room temperature and then extracted with 5 ml chloroform (65° C.). The mixture was cooled to room temperature and filtered through a Teflon filter (pore diameter: 0.2 μm). The insoluble residue was extracted again with 5 ml chloroform (65° C.) and filtered off. The solvent was removed by distillation. A product of 131 mg was obtained. The product obtained has a residual manganese content of 89 ppm (analysis by ICP-OES).

### Examples 16 to 18

Examples 16, 17 and 18 were carried out analogously to Examples 12, 13 and 14 respectively, using 5 wt % manganese (II) palmitate (Mn(Palm)₂) instead of manganese (IV) oxide. The results are summarized in Table 3.

### Example 19

Example 19 was carried ot analogously to Example 15, using 5 wt % manganese(II) palmitate (Mn(Palm)₂) instead of manganese(IV) oxide. 103 mg of product were obtained. The results are summarized in Tables 3 and 4.

### Example 20

Example 20 was carried out analogously to Example 13 whereby additionally 10 mg of N-hydroxyphthalimide (NHPI) were added. The results are summarized in Table 5.

### Example 21

200 mg of granulated HDPE (Purell GB 7250, LyondellBasell Industries, density: 0.952 g/cm³, Tₘ=134° C., crystallinity=62% (DSC), M_{W}=71.2 kg/mol) were weighed in a glass insert.

This glass insert was placed in a steel autoclave (with needle valve, manometer and bursting disk). This steel autoclave was then pressurized to 20.0 bar at room temperature using a compressor and then placed in a heating block preheated to 180° C. and heated for 4 hours. The start of the reaction time was represented by the insertion of the steel autoclave into the preheated heating block. After the reaction time had elapsed the steel autoclave was removed from the heating block the pressure was released and the gas phase was analyzed. (The results of this analysis are summarized in Table 4.) The molten cake was cooled to room temperature and then extracted with 5 ml chloroform (65° C.). The mixture was cooled to room temperature and filtered through a Teflon filter (pore diameter: 0.2 μm). The insoluble residue was extracted again with 5 ml chloroform (65° C.) and filtered off. The solvent was removed by distillation. 152 mg of product were obtained.

### Example 22

3000 mg of granulated HDPE (Purell GB 7250, LyondellBasell Industries, density: 0.952 g/cm³, Tₘ=134° C., crystallinity=62% (DSC), M_{W}=71.2 kg/mol) mixed with 10 wt % manganese (IV) oxide were weighed into a steel autoclave (with needle valve, manometer and bursting disk). This steel autoclave was then pressurized to 20.0 bar at room temperature using a compressor and subsequently placed in a heating block preheated to 160° C. and heated for 2 h. The start of the reaction time was represented by the insertion of the steel autoclave into the preheated heating block. After the reaction time had elapsed the steel autoclave was removed from the heating block and the pressure was released. The melt cake was then extracted with 400 ml 1,2-dichlorobenzene (150° C.). The mixture was cooled to room temperature and the supernatant solution was removed as completely as possible from the separated catalyst. The catalyst phase was centrifuged off (3500 rpm, 10 min). The insoluble catalyst residue was first extracted again hot (150° C.) with 45 ml 1,2-dichlorobenzene, then twice with 45 ml chloroform and centrifuged off. The residue was dried overnight at 5 mbar and 60° C.

Subsequently, 200 mg of granulated HDPE (Purell GB 7250, LyondellBasell Industries, density: 0.952 g/cm³, Tₘ=134° C., crystallinity=62% (DSC), M_{W}=71.2 kg/mol) mixed with 0.77% by weight of the previously separated catalyst were weighed in a glass insert. This glass insert was placed in a steel autoclave (with needle valve, manometer and bursting disk). This steel autoclave was then pressurized to 20.0 bar at room temperature using a compressor and then placed in a heating block preheated to 180° C. and heated for 4 hours. The start of the reaction time was represented by the insertion of the steel autoclave into the preheated heating block. After the reaction time had elapsed the steel autoclave was removed from the heating block the pressure released and the gas phase analyzed (the results of this analysis are summarized in Table 6.) The melt cake was cooled to room temperature and then extracted with 5 ml chloroform (65° C.). The mixture was cooled to room temperature and filtered through a Teflon filter (pore diameter: 0.2 μm). The insoluble residue was extracted again with 5 ml chloroform (65° C.) and filtered off. The solvent was removed by distillation. 109 mg of product were obtained.

TABLE 3
Comparison of the reactions carried out in
examples 12 to 19 with MnO₂ and Mn(Palm)₂

| | |
|---|---|
| Reaction time | Proportion of O₂ after the reaction [%] |

| [h] | MnO₂ | Mn(Palm)₂ |
|---|---|---|
| 1 | 17.0 | 11.8 |
| 2 | 8.0 | 6.1 |
| 3 | 5.6 | 3.2 |
| 4 | 3.4 | 2.5 |

TABLE 4
Comparison of reactions without catalyst,
with MnO₂ and with Mn(Palm)₂.

| | | Proportion of O₂ |
|---|---|---|
| Example | Catalyst | after the reaction [%] |
| 15 | MnO₂ | 3.4 |
| 19 | Mn(Palm)₂ | 2.5 |
| 21 | without catalyst | 9.1 |

The data in Table 4 shows that the catalyst significantly accelerates the reaction, with the homogeneous catalyst Mn(Palm)₂ and the heterogeneous catalyst MnO₂ exhibiting comparable activities. Without catalyst a significantly slower reaction is observed.

TABLE 5
Comparison of reactions with MnO₂ and
with or without the addition of NHPI.

| | | Proportion of O₂ |
|---|---|---|
| Example | Catalyst | after the reaction [%] |
| 12 | MnO₂ | 17.0 |
| 20 | MnO₂ + NHPI | 13.1 |

The data in Table 5 shows that NHPI bridges the initially lower activity of the heterogeneous catalyst MnO₂.

TABLE 6
Comparison of the reactions with fresh and recovered MnO₂.

| | | Proportion of O₂ |
|---|---|---|
| Example | Catalyst | after the reaction [%] |
| 15 | Fresh MnO₂ | 3.4 |
| 24 | Recovered Catalyst | 3.3 |

1. A process for the preparation of a water-insoluble mixture of a homologous series of a plurality of different linear α,ω-alkyl dicarboxylic acids having a carbon chain length of at least C₈, starting from polyethylene-containing mixtures by oxidative cleavage with an oxygen-containing reaction gas, comprising:
providing a polyethylene-containing mixture which has a total polyethylene content of at least 50% by mass with an HDPE content of at least 5.0% by mass, whereby the polyethylenes contained each have a weight-average molecular weight (Mw) of at least 20,000 g/mol, and
heating the mixture provided to a temperature in a range from above the melting point of the polyethylene-containing mixture to 300° C. in an oxygen-containing reaction gas consisting of at least 5% by volume of oxygen at a process pressure of at least 1 bar in the presence of at least one catalyst in order to oxidatively decompose the polyethylene-containing mixture,
wherein the resulting product mixture comprises a plurality of different linear α,ω-alkyl dicarboxylic acids with a carbon chain length of at least C8 and an acid number of at least 100 mg KOH/g.

2. The process according to claim 1, wherein the polyethylene-containing mixture employed has a secondary raw material content of at least 50% by mass.

3. The process according to claim 2, wherein the polyethylene-containing secondary raw material stream employed has a total polyethylene content of at least 90% by mass and a relative HDPE content of at least 50% by mass.

4. The process according to claim 1, wherein at least one catalyst selected from the group consisting of N-hydroxyphthalimide (NHPI), metals and metal salts is employed, wherein the metals are selected from the group of Mo, Ru, Rh, Pd, Ag, W, Re, Os, Ir, Pt and Au, and wherein the cation of the metal salts is selected from the group of Mn, Fe, Co, Ni, Cu, Zn, Cr, V, Ti, Ru, Rh, Pd, Ag, Mo, W, Re, Os, Ir, Pt and Au and the anion of the metal salts is at least one selected from the group consisting of laurate, myristate, palmitate, palmitoleate, stearate, oleate, erucate, naphthenate, acetate, acetylacetonate, chloride, bromide, iodide, nitrate, oxide, sulfate, carbonate and phosphate.

5. The process according to claim 1, wherein the catalyst comprises at least one catalyst which is not soluble in the product mixture.

6. The process according to claim 5, wherein the catalyst which is insoluble in the product mixture is selected from the group of transition metal oxides, noble metals, supported noble metals and supported metal salts.

7. The process according to claim 6, wherein the catalyst comprises a manganese oxide.

8. The process according to claim 5, wherein the catalyst comprises N-hydroxyphthalimide.

9. The process according to claim 1, wherein a catalyst mixture is used, consisting of at least two catalysts, wherein at least one of the catalysts comprises at least one catalyst which is not soluble in the product mixture and at least one catalyst which is soluble in the product mixture.

10. The process according to claim 9, wherein the catalyst soluble in the product mixture is selected from the group consisting of transition metal salts soluble in the product mixture and N-hydroxyphthalimide.

11. The process according to claim 5, wherein the catalyst which is not soluble in the product mixture is separated from the reaction mixture or the reaction product after the reaction with the oxygen-containing reaction gas.

12. The process according to claim 1, wherein the amount of the at least one catalyst is 0.0001 to 10.0% by mass, based on 100% by mass of the polyethylene-containing mixture employed.

13. The process according to claim 1, wherein the reaction is carried out in water or in an aqueous medium as reaction medium.

14. The process according to claim 1, wherein the product mixture is purified by at least one process selected from the group consisting of filtration, drying, precipitation, extraction, crystallization, fractional distillation, steam distillation and column chromatography.

15. The process according to claim 1, wherein at least 50 mol % of the detectable products obtained have a carbon chain length in the range of C₈ to C₃₄ as determined by gas chromatography using linear α,ω-alkyl dicarboxylic acids as reference substances.

16. A mixture of a homologous series of a plurality of different linear α,ω-alkyl dicarboxylic acids having a carbon chain length of at least C₈, obtainable by a process according to claim 1, wherein at least 50 mol % of the detectable products obtained have a carbon chain length in the range C₈ to C₃₄ as determined by gas chromatography using linear α,ω-alkyl dicarboxylic acids as reference substances.

17. A composition consisting essentially of a water-insoluble mixture of a homologous series of at least 10 different linear α,ω-alkyl dicarboxylic acids having a carbon chain length in the range from C₈ to C₃₄, the proportion of water-soluble compounds and compounds having a carbon chain length of C₃₅ or more being less than 10% by mass.

18. The mixture according to claim 16, wherein the average carbon chain length of the mixture, which is defined as the maximum of a distribution determined by gas chromatography using linear α,ω-alkyl dicarboxylic acids as reference substances, is ≥C₉.

19. The mixture according to claim 16, wherein the mixture contains at least 5% by mass of keto-functionalized and/or hydroxy-functionalized α,ω-alkyl dicarboxylic acids.

20. The mixture according to claim 16, wherein the mixture contains at least 5% by mass of α,ω-alkyl dicarboxylic acids with a chain length ≥C₁₉.

21. The mixture according to claim 16, wherein the mixture contains at least 10% by mass of α,ω-alkyl dicarboxylic acids with a chain length in the range C₁₉ to C₃₄.

22-25. (canceled)

26. A pure linear α,ω-alkyl dicarboxylic acid having a carbon chain length in the range from C₈ to C₃₄, an emulsifier, a water-based lubricant, or a biodegradable polyester prepared from the mixture of claim 16.

27. The process according to claim 7, wherein the catalyst comprises manganese dioxide.

28. The mixture according to claim 18, wherein the average carbon chain length of the mixture is in the range from C₁₂ to C₂₈.

29. The mixture according to claim 19, wherein the mixture contains 5 to 20% by mass of keto-functionalized and/or hydroxy-functionalized α,ω-alkyl dicarboxylic acids

30. The mixture according to claim 20, wherein the mixture contains 5 to 80% by mass of α,ω-alkyl dicarboxylic acids with a chain length ≥C₁₉.

31. The mixture according to claim 21, wherein the mixture contains 10 to 80% by mass of α,ω-alkyl dicarboxylic acids with a chain length in the range C₁₉ to C₃₄.
